(19) United States
(12) Patent Application Publication
(10) Pub. No.: US 2024/0264178 A1
(43) Pub. Date: Aug. 8, 2024

(54) METHOD FOR ANALYZING STEROID BY LIQUID CHROMATOGRAPH MASS SPECTROMETRY

(71) Applicants: SHIMADZU CORPORATION, Kyoto (JP); National Institute for Environmental Studies, Ibaraki (JP)

(72) Inventors: Hiroyuki NIWA, Kyoto-shi (JP); Aurore JAFFUEL, Kyoto-shi (JP); Jun WATANABE, Kyoto-shi (JP); Shoji NAKAYAMA, Tsukuba-shi (JP)

(73) Assignees: SHIMADZU CORPORATION, Kyoto (JP); National Institute for Environmental Studies, Ibaraki (JP)

(21) Appl. No.: 18/431,514

(22) Filed: Feb. 2, 2024

(30) Foreign Application Priority Data
Feb. 3, 2023 (JP) 2023-015154

(51) Int. Cl. G01N 33/74

(52) U.S. Cl. CPC G01N 33/743; G01N 2333/723

(57) ABSTRACT

In order to reduce the time required for an analysis of a steroid compound in a sample, a method for analyzing a steroid compound according to the present invention includes: a fractionating process for dividing one sample containing a steroid compound into a plurality of liquid fractions; a treating process for performing a different treatment on each of the plurality of liquid fractions; a mixing process for preparing a single mixed sample solution by mixing the plurality of liquid fractions after the treatment; and an analyzing process for analyzing a steroid compound contained in the mixed sample solution with a liquid chromatograph mass spectrometer.

### TECHNICAL FIELD

The present invention relates to a method for analyzing steroid compounds in a sample by using a liquid chromatograph mass spectrometer.

### BACKGROUND ART

“Steroid compound” is a general term for compounds having a structure called the “steroidal backbone” in which three six-membered rings and one five-membered ring are connected. Steroid compounds are known to exhibit various physiological activities depending on the type of side chain. A living organism contains various natural steroids, including steroid hormones (e.g., sex hormone or adrenal steroid), bile acids and cholesterol which is an important lipid for the formation of cell membranes. There are also many drugs developed from synthetic steroids. For example, dihydrotestosterone, which is one of the banned drugs whose unauthorized use often becomes a problem in athletic competitions, is a synthetic steroid.

Quantitative and qualitative measurements of steroids in living organisms have been conducted for the purpose of clinical diagnosis or analysis of pathological conditions, or for the testing of banned drugs. Liquid chromatograph mass spectrometry (LC/MS or LC/MS/MS) has been widely used for an analysis of steroids since the technique allows for a specific and collective analysis of various steroids present in a living organism.

The quantity of a steroid present in the body of a living organism considerably varies depending on the kind of steroid, properties of the living organism and other factors. For example, the amount of estrogen (female hormone), such as estradiol, in the body of an infant or a postmenopausal female is extremely small. In order to enable an accurate measurement of such a trace amount of steroid compound, a method for analyzing a steroid has been proposed in which only a steroid compound having a specific structure is derivatized among the steroid compounds in a sample (Patent Literature 1).

In the method disclosed in Patent Literature 1, a steroid compound having one hydroxyl group is derivatized by introducing an N-alkyl pyridinium group into the hydroxyl group in the steroid compound by using a derivatization reagent which specifically reacts with that steroid compound. In the case of a sample which contains a steroid compound having one hydroxyl group and a steroid compound having two or more hydroxyl groups, some of those steroid compounds will not be derivatized when the aforementioned derivatization reagent is added to the sample. The derivatization reagent will ultimately be an unwanted component for an LC/MS analysis of a steroid compound having two or more hydroxyl groups. Therefore, in the conventional method, the sample is divided into two specimens, and an LC/MS analysis is performed for each of the specimens, with one specimen pretreated with the derivatization reagent added and the other specimen pretreated without the derivatization reagent.

However, it is difficult to divide a small amount of sample into two or more specimens of equal quantity. Increasing the amount of sample to address this problem would lead to a corresponding increase in a physical burden for the subject from whom the sample is collected.

Conducting an LC/MS analysis for each of the two or more specimens also has the problem of an increase in the analyzing time.

### CITATION LIST

### Patent Literature

Patent Literature 1: JP 2003-161726 A

### SUMMARY OF INVENTION

### Technical Problem

The problem to be solved by the present invention is to shorten the period of time required for an analysis of a steroid compound contained in a sample.

### Solution to Problem

The present invention developed for solving the previously described problem is a method for analyzing steroid compounds in a sample, including:
a fractionating process for dividing one sample containing steroid compounds into a plurality of liquid fractions;
a treating process for performing a different treatment on each of the plurality of liquid fractions;
a mixing process for preparing a single mixed sample solution by mixing the plurality of liquid fractions after the treatment; and
an analyzing process for analyzing a steroid compound contained in the mixed sample solution with a liquid chromatograph mass spectrometer.

### Advantageous Effects of Invention

According to the present invention, when there is a plurality of steroid compounds contained in one sample, the sample can be divided into a plurality of liquid fractions so that those steroid compounds are divided into the liquid fractions, and a different treatment can be performed on those steroid compounds in each liquid fraction. The liquid fractions each of which contains an already treated steroid compound are further mixed together to form a single solution, on which a liquid chromatograph mass spectrometric analysis is performed. Therefore, the period of time required for the analysis can be shortened.

### BRIEF DESCRIPTION OF DRAWINGS

FIG. 1 is a schematic configuration diagram of one embodiment of a high-performance liquid chromatograph mass spectrometer for carrying out a method for analyzing a steroid compound according to the present invention.

FIG. 2 is a diagram showing a derivatization reaction of estradiol.

FIG. 3 is a flow chart showing a procedure for preparing a mixed sample solution.

### DESCRIPTION OF EMBODIMENTS

In the method for analyzing a steroid compound in a sample according to the present invention, one sample containing one or more steroid compounds is initially divided into a plurality of liquid fractions, and a different treatment is performed on each of the plurality of liquid fractions. Subsequently, the plurality of treated liquid fractions are mixed together to form a single mixed sample solution, and the steroid compounds contained in the mixed sample solution is analyzed with a liquid chromatograph mass spectrometer.

The sample to be the target in the previously described analyzing method may be a sample of tissue, blood, urine or other substances of biological origin suspended in or diluted with water, organic solvent or a mixture of water and organic solvent. The substance of biological origin can be collected from a patient suffering from a specific disease or from a healthy individual.

The steroid compounds contained in a sample may be natural steroids produced in a living organism or artificially synthesized synthetic steroids. Any kind of steroid compound can be the target of the analysis according to the present invention. A steroid compound contained in the mixed sample solution may be a steroid compound which is substantially identical to the steroid compound originally contained in the sample and has been derivatized by a predetermined treatment.

In the fractionating process in the previously described analyzing method, one sample should ideally be divided into fractions in such a manner that each of the liquid fractions contains a steroid compound, although there may be the case where some of the liquid fractions resulting from the fractionation contains no steroid compound.

In the treating process, a treatment suited for a steroid compound which is expected to be contained in the liquid fraction is performed for each of the liquid fractions obtained in the fractionating process.

Typically, in the fractionating process, the sample may be divided into two liquid fractions, and the treating process may be performed in such a manner that the treatment performed on one liquid fraction includes a derivatization treatment for a steroid compound, while the treatment performed on the other liquid fraction does not include the derivatization treatment.

Accordingly, in the present case, the fractionation of one sample is performed under a condition under which a steroid compound that requires derivatization and another steroid compound that requires no derivatization will be individually contained in separate liquid fractions.

In the mixing process, it is preferable that the single mixed sample solution prepared by mixing the plurality of liquid fractions after the treatment contains an organic acid or an organic-acid-salt buffer solution as a solvent.

In general, organic acids are carboxylic acids, including acetic acid, formic acid, oxalic acid, lactic acid, tartaric acid, citric acid and trifluoroacetic acid. As for the aforementioned organic-acid-salt buffer solution, for example, an ammonium formate buffer solution or ammonium acetate buffer solution can be used. There is also no specific limitation on the organic solvent; typically, acetonitrile can be used.

Thus, as one mode of the present invention, the mobile phase may be a mixed solution of an ammonium formate buffer solution and acetonitrile. In that case, it is preferable to perform a gradient analysis in which the concentration of the ammonium formate is increased with the passage of time.

For example, ammonium formate contains a positively charged ammonium ion and a negatively charged formic ion, thereby contributing to the action of retaining a halogen-oxide-acid ion in a sample by the ion exchange function of the stationary phase in the column of the liquid chromatograph. Additionally, organic acid salts, including ammonium formate, are volatile salts and hence unlikely to cause the problem of deposition (or the like) when introduced into an ESI ion source, for example, of the mass spectrometer. On the other hand, organic solvents, including acetonitrile, are polar solvents and contribute to the hydrophobic interaction (i.e., the reversed-phase function) of the stationary phase in the column, as well as to an efficient ionization of sample molecules in an ESI ion source, for example, of the mass spectrometer.

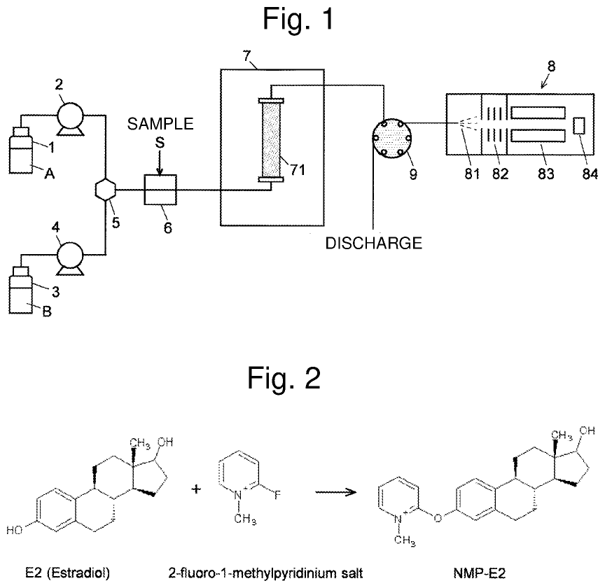

FIG. 1 is a schematic configuration diagram of a high-performance liquid chromatograph mass spectrometer (HPLC/MS) as one example of the mass spectrometer to be used in the analyzing process in the method for analyzing a steroid compound.

In FIG. 1, a first liquid supply pump 2 draws mobile phase A from a first mobile phase container 1 and sends it at a predetermined flow rate, while a second liquid supply pump 4 draws mobile phase B from a second mobile phase container 3 and sends it at a predetermined flow rate. The mobile phases A and B are mixed together in a mixer 5 and sent to a column oven 7 via an injector 6. In the injector 6, a liquid sample as the analysis target is injected into the mobile phase through a micro syringe (or the like). Being carried by the flow of the mobile phase, the liquid sample is sent into a column 71 in the column oven 7. While flowing through the column 71, the various components in the sample are separated from each other and exit from the exit end of the column 71, having temporal differences.

The eluate from the column 71 is sent through a switching valve 9 to a mass spectrometer 8 as the detector and is sprayed from a spray nozzle of an ESI ion source 81 into the ambience of substantially atmospheric pressure, whereby the component molecules contained in the eluate are ionized. The generated ions are converged by an ion lens 82 and are separated from each other by a quadrupole mass filter 83 according to their mass-to-charge ratios, to arrive at and be detected by an ion detector 84. The kind of component contained in the eluate, i.e., the kind of component to be subjected to the mass spectrometry, changes with the passage of time. The quadrupole mass filter 83 is operated in a selected ion monitoring (SIM) mode so as to detect one or more ions having a previously specified mass-to-charge ratio or ratios. Accordingly, the detection signal obtained with the ion detector 84 reflects each individual component. In a data processing unit (not shown), a mass chromatogram corresponding to a halogen-oxide-acid compound as the target component is created based on the detection signal, and a qualitative and/or quantitative analysis of the target component is performed based on a peak which appears in the chromatogram.

The ion source of the mass spectrometer 8 is not limited to an ESI ion source; an ion source employing the technique of atmospheric pressure chemical ionization (APCI) or atmospheric photoionization (APPI) may also be used. The mass separator is not limited to a quadrupole mass filter; it may be a different type of device, such as a time-of-flight mass analyzer. A mass spectrometer capable of an MS/MS or MSⁿ analysis may also be used, such as a triple quadrupole mass spectrometer.

As for the liquid chromatograph to be connected to the mass spectrometer, there are various liquid chromatographs available, including a nano-flow liquid chromatograph, micro-flow liquid chromatograph, high-performance liquid chromatograph and ultra high-performance liquid chromatograph. In short, an appropriate mass spectrometer and an appropriate liquid chromatograph can be used in combination according to the kind and nature of the sample and the steroid compound to be analyzed.

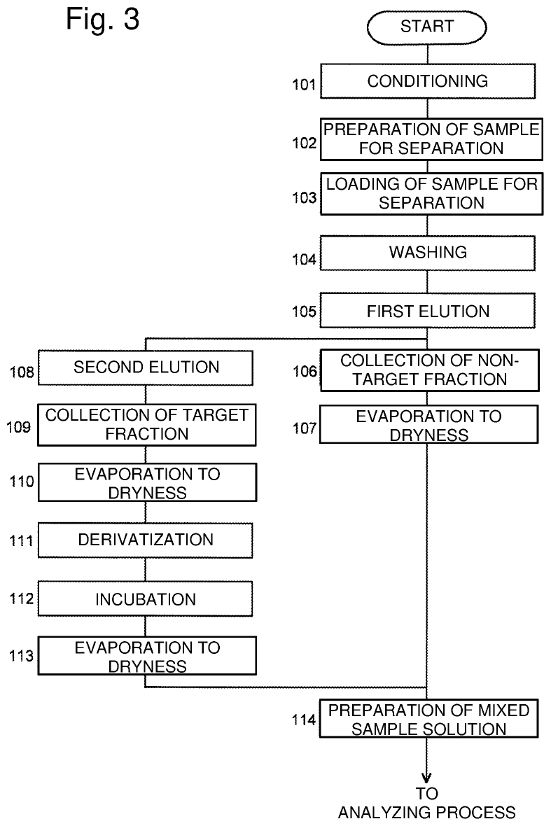

A procedure for preparing a mixed sample solution in advance of an analysis of a steroid compound in a biological specimen is hereinafter described with reference to FIG. 3. The following procedure is an example of a procedure in the case where the analysis target is estradiol, which is a steroid compound. The chemical structure of estradiol is shown in FIG. 2. Estradiol is a kind of female hormone (estrogen) produced in the adrenal cortex. It has two hydroxyl groups and one methyl group. It is hereinafter assumed that estradiol is derivatized (into NMP-E2) by introducing a 2-fluoro-1-methylpyridinium group into one of the two hydroxyl groups before the analysis is performed with the mass spectrometer 8.

### Solid-Phase Extraction

The steroid compounds contained in the sample are divided into a plurality of liquid fractions by using a solid-phase extraction column (also called a “solid-phase extraction cartridge”). A solid-phase extraction column consists of a cartridge packed with a filler as the solid phase. The material to be adopted as the filler is a material by which a steroid compound as the analysis target can be separated from the other steroid compounds among the steroid compounds contained in the sample solution. For example, a product offered by Waters Corporation (product name: Oasis MAX) may be used, which is a mixture of a reversed-phase type of filler and an anion exchange type of filler.

### Conditioning of Solid-Phase Extraction Column: Step 101

A conditioning is performed in order to activate the functional groups of the filler in the solid-phase extraction by wetting the filler. Examples of the conditioning liquid used for the conditioning include methanol, a mixed solution of 100-mmol/L ammonium bicarbonate/25% ammonium water (50:2, v/v), 90% acetonitrile, and a mixed solution of water/methanol/acetic acid (90:10:1, v/v/v). The operation of wetting the filler with the conditioning liquid may be performed only one time or be repeated a plurality of times.

### From Separation of Steroid Compound in Biological Specimen to Preparation of Mixed Sample Solution: Steps 102 through 114

In advance of the separation of steroid compounds in a biological specimen, a sample for separation is prepared (Step 102). Initially, an amount of methanol and a mixture of water/methanol/acetic acid with a volume ratio of 90:10:1 (v/v/v) are put into a container in which the biological specimen has been contained. The content is subsequently stirred with a vortex mixer, whereby the sample for separation is obtained. As for the biological specimen, a sample of tissue, blood, urine, bile or other substances collected from a subject may be used.

Subsequently the sample for separation is passed through the filler in the solid-phase extraction column which has been conditioned (“loading”: Step 103). Next, an appropriate kind of washing liquid selected according to the polarity of the steroid compound and that of unnecessary components contained in the sample for separation is passed through the filler (“washing”: Step 104). The unnecessary components adsorbed to the filler are thereby discharged from the solid-phase extraction column. Examples of the available washing liquids include 25% methanol, 100-mmol/L ammonium bicarbonate/25% ammonia water (50:2(v/v)), and water/25% ammonia water (95:5(v/v)).

Next, an eluent is passed through the filler in the solid-phase extraction column to elute some of the steroid compounds retained on the filler (“first elution”: Step 105). In this case, either an eluent which allows the target steroid compound to remain retained on the filler, or an eluent which causes the target steroid compound to be eluted from the filler, is used. It is hereinafter assumed that an eluent which allows estradiol as the target steroid compound to remain retained on the filler is used (this eluent is hereinafter called the “first eluent”). An example of the first eluent of this type is 90% acetonitrile. As a result, the fraction containing the target steroid compound (“target fraction”) remains in the solid-phase extraction column, while the fractions with no target substance contained (which are hereinafter called the “non-target fractions”) are eluted from the solid-phase extraction column. It should be noted that the first eluent should be appropriately selected so that estradiol will not be contained in the non-target fractions, whereas steroid compounds other than estradiol may be contained in the target fraction.

Subsequently, the eluted non-target fractions are collected (Step 106) and evaporated to dryness at 40 degrees Celsius by using nitrogen gas (Step 107).

Meanwhile, an eluent (“second eluent”) different from the first eluent is passed through the filler in the solid-phase extraction column in which the target fraction is retained on the filler (“second elution”: Step 108) to elute and collect the target fraction from the filler (Step 109). As for the second eluent, for example, a solution prepared so that acetonitrile, water and acetic acid are contained at a ratio of 45:25:30 can be used. As for the acetic acid, for example, a 30% acetic acid is used.

The collected target fraction is evaporated to dryness (Step 110), and a derivatization reagent is added to it (Step 111). The derivatization reagent is a mixture of 2-fluoro-1-methylpyridinium p-toluenesulfonate (FMP-TS), triethylamine and acetonitrile. After that, the target fraction with the derivatization reagent added is warmed at 40 degrees Celsius for 15 minutes (Step 112), whereby estradiol is derivatized. The target fraction which has been derivatized is once more evaporated to dryness (Step 113). The dried residue of the target fraction and that of the non-target fractions obtained Step 107 are mixed together, to which 100 μL of water/acetonitrile (4:1(v/v)) is added to prepare a mixed sample solution (Step 114).

### Mass Spectrometric Analysis of Steroid Compounds

The mixed sample solution obtained in Step 114 is introduced into the previously described HPLC/MS, with which all steroid compounds in the mixed sample solution are collectively analyzed. In most cases, the content of estradiol is lower than those of the other steroid compounds. Therefore, conventionally, when estradiol and other steroid compounds are collectively subjected to a mass spectrometric analysis, the measurement for estradiol will be rather inaccurate and poorly precise. By comparison, in the previously described embodiment, the content of estradiol can be measured with a high level of accuracy since estradiol is derivatized before being collectively subjected to the mass spectrometric analysis along with the other steroid compounds.

In the analysis in which the mixed sample solution is introduced into the HPLC/MS, it is preferable to operate the switching valve 9 so as to prevent the eluate from being sent to the mass spectrometer 8 during the period of time in which an unreacted derivatization reagent in the mixed sample solution exits from the column 71. Allowing the unreacted derivatization reagent to be introduced into the mass spectrometer 8 along with a steroid compound would cause a deterioration in the analysis sensitivity for the target component due to an increase in the background level or a contamination of the mass spectrometer 8. Such problems can be prevented by the previously described switching operation.

### Various Modes

A person skilled in the art can understand that the previously described illustrative embodiment is a specific example of the following modes of the present invention.

(Clause 1) One mode of the present invention is a method for analyzing a steroid compound in a sample, including:
a fractionating process for dividing one sample containing a steroid compound into a plurality of liquid fractions;
a treating process for performing a different treatment on each of the plurality of liquid fractions;
a mixing process for preparing a single mixed sample solution by mixing the plurality of liquid fractions after the treatment; and
an analyzing process for analyzing a steroid compound contained in the mixed sample solution with a liquid chromatograph mass spectrometer.

By the method for analyzing a steroid compound according to Clause 1, when there is a plurality of steroid compounds contained in one sample, those steroid compounds can be divided into a plurality of liquid fractions, and a different treatment can be performed on a steroid compound in each liquid fraction. The liquid fractions each of which contains an already treated steroid compound are further mixed together to form a single solution, on which a liquid chromatograph mass spectrometric analysis is performed. Therefore, the period of time required for the analysis can be shortened.

(Clause 2) In the method for analyzing a steroid compound according to Clause 2, which is one mode of the method for analyzing a steroid compound according to Clause 1, the treatment performed on one of the plurality of liquid fractions in the treating process includes a derivatization treatment for a steroid compound having a specific structure, while the treatment performed on at least one of the remaining liquid fractions does not include the aforementioned derivatization treatment.

By the method for analyzing a steroid compound according to Clause 2, the measurement accuracy for a steroid compound having a specific structure can be improved. Derivatization treatments include a treatment for replacing a specific structure by a different structure for, and a treatment for replacing one or more elements in a specific structure by their isotopes.

(Clause 3) In the method for analyzing a steroid compound according to Clause 3, which is one mode of the method for analyzing a steroid compound according to Clause 1:
the one sample is divided into two liquid fractions in the fractionating process; and
the treatment performed on one of the two liquid fractions in the treating process includes a derivatization treatment of estradiol, while the treatment performed on the other liquid fraction does not include the aforementioned derivatization treatment.

By the method for analyzing a steroid compound according to Clause 3, even when the amount of estradiol contained in the sample is extremely small, the measurement of the estradiol can be performed with a high level of accuracy.

(Clause 4) In the method for analyzing a steroid compound according to Clause 4, which is one mode of the method for analyzing a steroid compound according to one of

Clauses 1-3, the fractionating process includes dividing the sample into a plurality of liquid fractions by introducing the one sample into a solid-phase extraction column.

(Clause 5) In the method for analyzing a steroid compound according to Clause 5, which is one mode of the method for analyzing a steroid compound according to Clause 4, the solid-phase extraction column is an ion exchange type of solid-phase extraction column.

(Clause 6) In the method for analyzing a steroid compound according to Clause 6, which is one mode of the method for analyzing a steroid compound according to Clause 5, the solvent of the mixed sample solution contains an organic acid or an organic acid salt.

### REFERENCE SIGNS LIST

1 . . . First Mobile Phase Container
2 . . . First Liquid Supply Pump
3 . . . Second Mobile Phase Container
4 . . . Second Liquid Supply Pump
5 . . . Mixer
6 . . . Injector
7 . . . Column
8 . . . Mass Spectrometer
81 . . . ESI Ion Source
82 . . . Ion Lens
83 . . . Quadrupole Mass Filter
84 . . . Ion Detector

1. A method for analyzing a steroid compound in a sample, comprising:
a fractionating process for dividing one sample containing a steroid compound into a plurality of liquid fractions;
a treating process for performing a different treatment on each of the plurality of liquid fractions;
a mixing process for preparing a single mixed sample solution by mixing the plurality of liquid fractions after the treatment; and
an analyzing process for analyzing a steroid compound contained in the mixed sample solution with a liquid chromatograph mass spectrometer.

2. The method for analyzing a steroid compound in a sample according to claim 1, wherein the treatment performed on one of the plurality of liquid fractions in the treating process includes a derivatization treatment for a steroid compound having a specific structure, while the treatment performed on at least one of the remaining liquid fractions does not include the aforementioned derivatization treatment.

3. The method for analyzing a steroid compound in a sample according to claim 1, wherein:
the one sample is divided into two liquid fractions in the fractionating process; and
the treatment performed on one of the two liquid fractions in the treating process includes a derivatization treatment of estradiol, while the treatment performed on the other liquid fraction does not include the aforementioned derivatization treatment.

4. The method for analyzing a steroid compound in a sample according to claim 1, wherein the fractionating process includes dividing the sample into a plurality of liquid fractions by introducing the one sample into a solid-phase extraction column.

5. The method for analyzing a steroid compound in a sample according to claim 4, wherein the solid-phase extraction column is an ion exchange type of solid-phase extraction column.

6. The method for analyzing a steroid compound in a sample according to claim 5, wherein the solvent of the mixed sample solution contains an organic acid or an organic acid salt.
